(19) United States
(12) Patent Application Publication
(10) Pub. No.: US 2023/0293814 A1
(43) Pub. Date: Sep. 21, 2023

(54) Dressing-Integrated Tubing Occlusion

(71) Applicant: Becton, Dickinson and Company, Franklin Lakes, NJ (US)

(72) Inventors: John M. Lackey, West Valley City, UT (US); Megan S. Scherich, Salt Lake City, UT (US)

(21) Appl. No.: 18/122,965

(22) Filed: Mar. 17, 2023

Related U.S. Application Data
(60) Provisional application No. 63/321,938, filed on Mar. 21, 2022.

(51) Int. Cl. A61M 5/168; A61M 39/28

(52) U.S. Cl. CPC A61M 5/16813; A61M 39/28; A61M 2025/0266

(57) ABSTRACT

Provided herein is an intravascular catheter assembly, including a catheter adapter having a distal end, a proximal end, a lumen arranged between and in fluid communication with the distal end and the proximal end, a catheter arranged at the distal end of the catheter adapter and in fluid communication with the lumen, and a side port in fluid communication with the lumen. The assembly further includes a fluid conduit having a distal end and a proximal end, the distal end of the fluid conduit coupled to the side port, a connector arranged at the proximal end of the fluid conduit, and an occlusion device configured to stop flow through the fluid conduit.

### CROSS-REFERENCE TO RELATED APPLICATION

The present application claims priority to U.S. Provisional Application Ser. No. 63/321,938, entitled “Dressing-Integrated Tubing Occlusion”, filed Mar. 21, 2022, the entire disclosure of which is hereby incorporated by reference in its entirety.

### BACKGROUND OF THE INVENTION

### Field of the Invention

The present disclosure relates generally to intravenous (IV) catheter assemblies and, more specifically, to IV catheter assemblies with occlusion devices to stop flow through the catheter adapter and allow for replacement of various components.

### Description of Related Art

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of intravenous (IV) catheter device includes a catheter that is over-the-needle. As its name implies, the catheter that is over-the-needle may be mounted over an introducer needle having a sharp distal tip. The IV catheter device may include a catheter adapter, the catheter extending distally from the catheter adapter, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

IV catheter devices can include an extension set with a plurality of ports and/or connectors for administering or withdrawing fluids from the patient, and/or flushing the catheter device. One or more of these ports and/or connectors may be replaceable; however, there is currently no way to occlude the extension set during such a replacement procedure. Thus, a need exists in the art for an IV catheter device that allows for simple occlusion of tubing associated therewith.

### SUMMARY OF THE INVENTION

Provided herein is an intravascular catheter assembly, including a catheter adapter having a distal end, a proximal end, a lumen arranged between and in fluid communication with the distal end and the proximal end, a catheter arranged at the distal end of the catheter adapter and in fluid communication with the lumen, and a side port in fluid communication with the lumen. The assembly further includes a fluid conduit having a distal end and a proximal end, the distal end of the fluid conduit coupled to the side port, a connector arranged at the proximal end of the fluid conduit, and an occlusion device configured to stop flow through the fluid conduit.

In certain configurations, the intravascular catheter assembly includes a dressing having a bottom surface configured to contact a patient's skin and a top surface. The occlusion device may be coupled to the top surface of the dressing. Optionally, the occlusion device is a clamp including a base and first and second arms extending substantially perpendicularly from the base, the base and the first and second arms defining a channel configured to receive the fluid conduit. The clamp may be formed of an elastomeric material.

In certain configurations, the first arm and the second arm are resiliently biased toward the channel. A diameter of the channel may be less than a diameter of the fluid conduit, such that when the fluid conduit is received by the channel, fluid flow through the fluid conduit is slowed and/or stopped.

The occlusion device may be a clamp including a base and a button defining a channel therebetween, the channel configured to receive the fluid conduit and the button configured to be moveable into the channel, wherein moving the button into the channel causes the clamp to occlude the fluid conduit, thereby slowing and/or stopping fluid flow through the fluid conduit. The button may be biased away from the channel. Optionally, the button may be biased away from the channel by a spring.

In other configurations, the occlusion device is a clamp including a base having a first end and a second end, a cover having a first end and a second end, the first end of the cover hingedly connected to the first end of the base, and a latching mechanism configured to secure the second end of the base to the second end of the cover. The second end of the base may be secured to the second end of the cover, and the cover and the base define a channel configured to hold the fluid conduit therein. A diameter of the channel may be less than a diameter of the fluid conduit, such that when the fluid conduit is held within the channel, fluid flow through the fluid conduit is slowed and/or stopped.

In still other configurations, the occlusion device is a slide clamp including a channel having at least one portion with a diameter less than a diameter of the fluid conduit and at least one other portion with a diameter equal to or greater than the diameter of the fluid conduit. The occlusion device may be arranged between the side port and the connector.

In still other configurations, the occlusion device is a valve. The valve may be arranged in the connector. Optionally, the valve includes a manual actuator. The connector may be a t-connector or a y-connector.

In certain configurations, the intravascular catheter assembly further includes a needleless access connector coupled to the connector. The needleless access connector may be releasably coupled to the connector.

Also provided herein is an intravascular catheter assembly, including a catheter adapter having a distal end, a proximal end, a lumen arranged between and in fluid communication with the distal end and the proximal end, a catheter arranged at the distal end of the catheter adapter and in fluid communication with the lumen, and a side port in fluid communication with the lumen. The assembly further includes a fluid conduit having a distal end and a proximal end, the distal end of the fluid conduit coupled to the side port, a connector arranged at the proximal end of the fluid conduit, and a stabilization device configured to receive the fluid conduit therein.

The intravascular catheter assembly may further include a dressing having a bottom surface configured to contact a patient's skin and a top surface. The stabilizing mechanism may be coupled to the top surface of the dressing. Optionally, the stabilizing mechanism may include a base and first and second arms extending substantially perpendicularly from the base, the base and the first and second arms defining a channel configured to receive the fluid conduit.

In certain configurations, the clamp is formed of an elastomeric material. Optionally, the first arm and the second arm are resiliently biased toward the channel. In certain configurations, the stabilization device comprises a base and first and second arms extending substantially perpendicularly from the base, wherein each arm comprises, at an end opposite of the base, a flange, such that the flanges, the first arm, the second arm, and the base define a channel configured to receive the fluid conduit.

### BRIEF DESCRIPTION OF THE DRAWINGS

Example embodiments will be described and explained with additional specificity and detail through the use of the accompanying drawings in which:

FIG. 1A is an upper perspective view of an example catheter assembly, according to some non-limiting embodiments or aspects;

FIG. 1B is a cross-sectional view of the catheter assembly of FIG. 1A, according to some non-limiting embodiments or aspects;

FIG. 2 is an upper perspective view of another example catheter assembly, according to some non-limiting embodiments or aspects;

FIG. 3 is an upper perspective view of another example catheter assembly, according to some non-limiting embodiments or aspects;

FIG. 4 is an upper perspective view of another example catheter assembly, according to some non-limiting embodiments or aspects;

FIGS. 5A-5F are side and top views of occlusion devices for use with catheter assemblies as described herein, according to some non-limiting embodiments or aspects; and

FIG. 6 is a side view of a stabilization device for use with catheter assemblies as described herein, according to some non-limiting embodiments or aspects.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

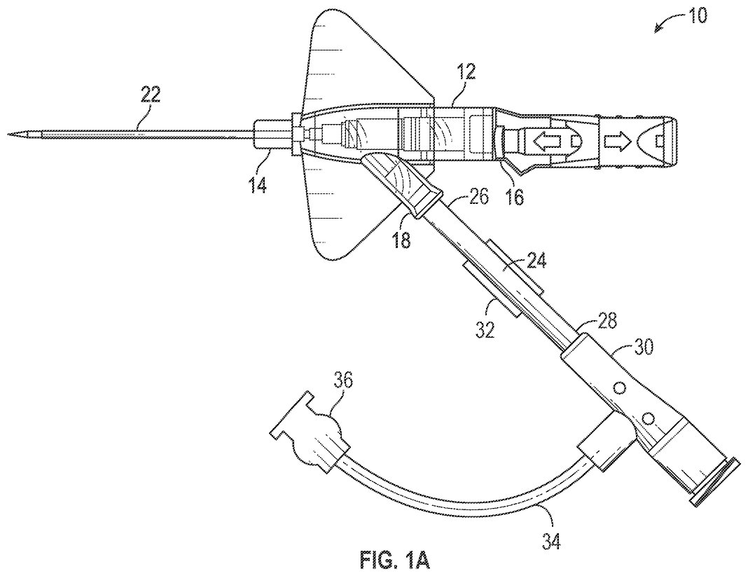

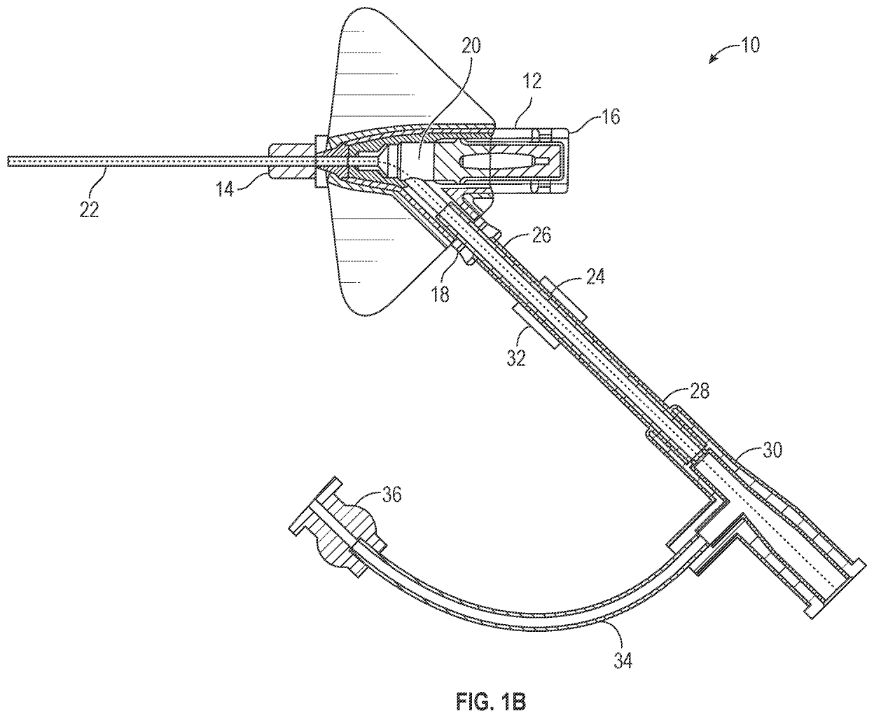

Referring now to FIGS. 1A and 1B, shown is a non-limiting embodiment of a catheter assembly 10, which may include a catheter adapter 12, which may include a distal end 14 and a proximal end 16. In some embodiments, the catheter adapter 12 may include a side port 18 disposed between the distal end 14 and a proximal end 16. In some embodiments, the first catheter adapter 12 may include a lumen 20 extending through the distal end 14 and the proximal end 16. FIG. 1B shows a needle hub and introducer needle removed, according to some embodiments or aspects, and also shows a cross-sectional view, such that lumen 20 is visible.

In some non-limiting embodiments or aspects, the first catheter assembly 10 may include a first catheter 22 extending from the distal end 14. In some embodiments, the first catheter 22 may include a peripheral intravenous catheter, a midline catheter, or a peripherally-inserted central catheter. Catheter 22 may be formed of any suitable material and may be of any useful length, as known to those of skill in the art. In some non-limiting embodiments or aspects, the first catheter assembly 10 may include fluid conduit 24 extending from the side port 18. Fluid conduit 24 may be formed of any suitable material known to those of skill in the art, and may have a distal end 26 and a proximal end 28, and fluid conduit may be coupled, at distal end 26 thereof, to side port 18. In some non-limiting embodiments or aspects, a connector 30 may be coupled to a proximal end 28 of fluid conduit 24.

In some non-limiting embodiments or aspects, catheter assembly 10 may include an extension set including extension line 34. Extension sets are known to those of skill in the art and are commercially available from, for example, Becton, Dickinson and Company under the tradenames MAXPLUS, MAXZERO, NEUTRACLEAR, Q-SYTE, and SMARTSITE. In some non-limiting embodiments or aspects, extension line 34 may include a luer connection 36 at an end thereof. In some non-limiting embodiments or aspects, the extension set may include a clamp 40, to allow for occlusion of extension line 34. Clamp 40 and extension line 34 may be formed of any suitable materials known to those of skill in the art.

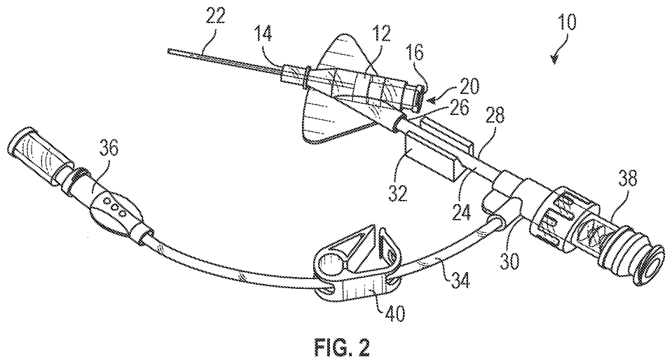

With continuing reference to FIGS. 1A and 1B, as well as FIG. 2, catheter assembly 10 may include an occlusion device 32 for slowing and/or stopping fluid flow through fluid conduit 24. As used herein, “occlude” or “occlusion” means at least a slowing of fluid flow through a conduit, such as fluid conduit 24, and includes within its scope complete cessation of flow through an occluded portion of a conduit. Various arrangements of an occlusion device 32 are described below and exemplified in the attached drawings. Arrangement of an occlusion device 32 along fluid conduit 24 can allow for cessation of flow of fluid, such as blood, from lumen 20 to connector 30. Such an occlusion device can allow a user, such as a healthcare professional, to replace one or more components of catheter assembly 10, such as a needleless access connector 38, as shown in FIG. 2. Needleless access connectors are known to those of skill in the art and are commercially available from, for example, Becton, Dickinson and Company under the tradenames SMARTSITE and Q-SYTE.

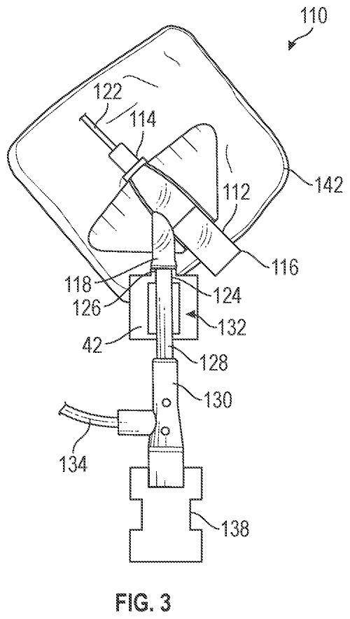

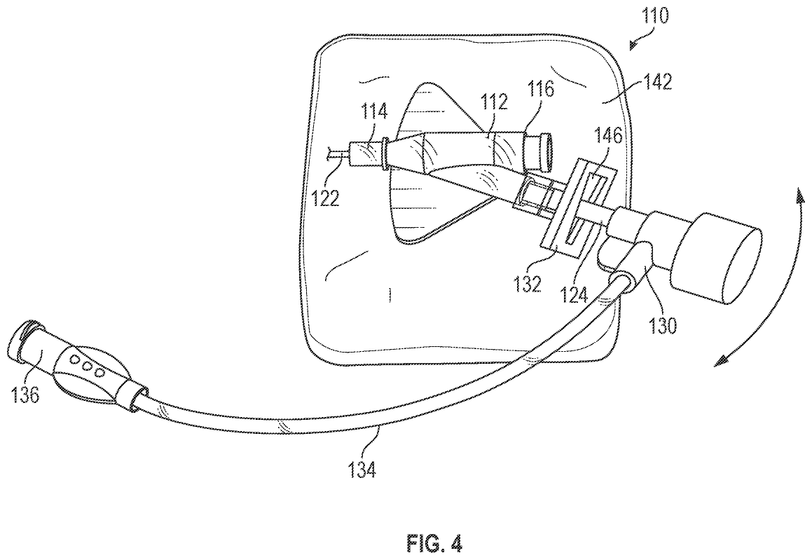

In some non-limiting embodiments or aspects, for example as shown in FIG. 3, catheter assembly 110, including catheter adapter 112, side port 118, catheter 122, fluid conduit 124, connector 130, extension set including extension line 134, needleless access connector 138, and/or occlusion device 132, may be secured to a patient at a site of insertion of catheter 122 with a dressing 142. Suitable dressings are known to those of skill in the art, and can include, for example, a gauze component as well as a clear film component, to cover the site of insertion, protecting the puncture from microorganisms in the ambient environment and/or securing the catheter assembly 110 in place to prevent a patient from dislodging the catheter 122. In some non-limiting embodiments or aspects, occlusion device 132 may be coupled to a dressing, e.g. dressing 142, for example as shown in FIG. 4, which can, in some embodiments, cover some or all of catheter assembly 110. In some non-limiting embodiments or aspects, occlusion device 132 may be coupled to either a gauze component and/or a clear film component of a dressing. In some non-limiting embodiments or aspects, dressing 142 may be configured such that a slit or other type of opening is provided in a gauze component and/or clear film component, allowing fluid conduit to pass through the dressing, and an occlusion device 232 as described herein can be arranged on a surface of dressing opposite a patient-contacting surface. In non-limiting embodiments, occlusion device may be coupled to a separate dressing, for example as shown in FIG. 3.

With reference to FIG. 4, shown is a non-limiting embodiment or aspect of a catheter assembly 110 according to non-limiting embodiments or aspects. As described previously, catheter assembly 110, including catheter adapter 112, side port 118, catheter 122, fluid conduit 124, connector 130, extension set including extension line 134, needleless access connector 138, and/or occlusion device 132, may be secured to a patient at a site of insertion of catheter 122 with a dressing 142. In the non-limiting embodiment or aspects shown in FIG. 4, occlusion device 132 is a clamp, such as a slide clamp, having a channel 146 through which fluid conduit 124 is received. In some non-limiting embodiments or aspects, channel 146 is not of a uniform diameter along the entire length thereof, such that fluid conduit 124 may be occluded by sliding fluid conduit 124 into a narrow portion of channel 146, thereby impinging on a wall of fluid conduit 124 and occluding flow therethrough. In non-limiting embodiments or aspect, occlusion device 132, in the form of a clamp, is coupled to dressing 142, which may, in some non-limiting embodiments or aspects, stabilize the clamp, making occlusion of fluid conduit 124 easier to achieve (e.g., with only one hand).

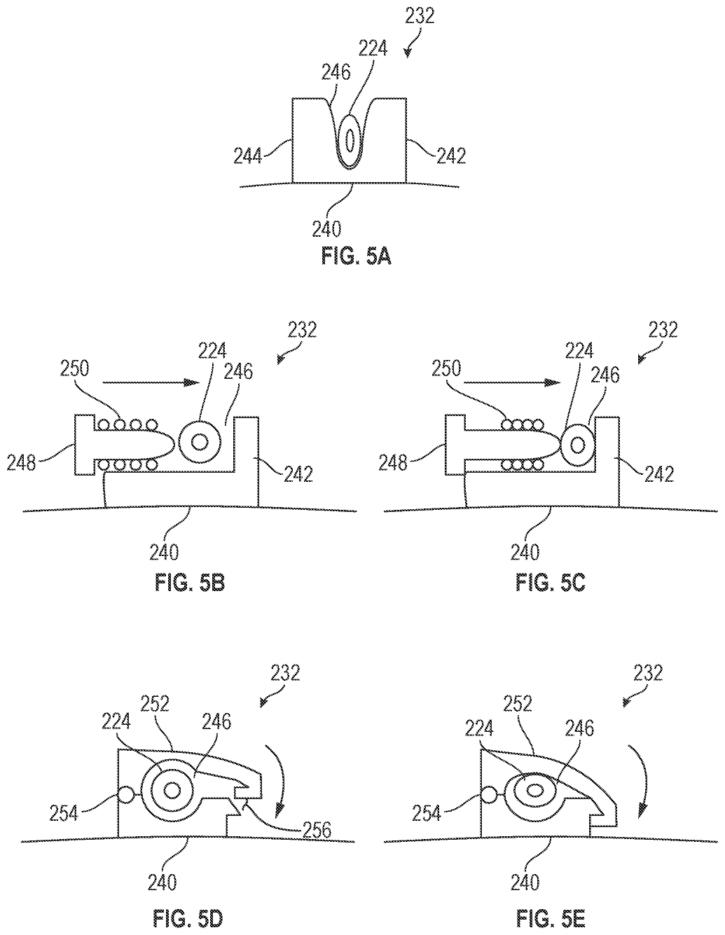

Turning to FIGS. 5A-5F, shown are various non-limiting embodiments or aspects of occlusion devices useful for catheter assemblies as described herein. With regard to FIG. 5A, shown is a non-limiting embodiment or aspect of occlusion device 232 in which fluid conduit 224 is held via interference fit. In the illustrated embodiment or aspect, occlusion device 232 may include a base 240 and first and second arms 242, 244 extending from base 240. In some non-limiting embodiments or aspects, first and second arms 242, 244 may extend substantially perpendicularly from base 240, optionally substantially parallel to one another. Base 240 and first and second arms 242, 244 define a channel 246 configured to hold fluid conduit 224 by, for example, interference fit. Occlusion device 232 as shown in FIG. 5A may be formed of any useful material, and in some non-limiting embodiments or aspects, is formed of a resilient material for example an elastomeric material, to allow for separation of arms 242, 244 (e.g., radially outward away from channel 246) to allow fluid conduit 224 to be introduced into, and removed from channel 246. In some non-limiting embodiments or aspects, channel 246 may have, along at least a portion of a length thereof, a diameter that is smaller than a diameter of fluid conduit 224, such that when fluid conduit 224 is received within channel 246, one or more of base 240 and arms 242, 244 impinge on a wall of fluid conduit 224 and occlude flow therethrough.

With reference to FIGS. 5B and 5C, shown is a non-limiting embodiment or aspect of occlusion device 232, in which fluid conduit 224 is occluded by virtue of being compressed by button 248. In the illustrated non-limiting embodiment or aspect, occlusion device 232 includes a base 240, first arm 242, and button 248, which is resiliently biased in a direction away from a channel 246 formed by base 240, first arm 242, and button 248. Button 248 may be biased by a resilient biasing element 250, which may be an elastomeric component, a spring, or other like mechanism that exerts a force on button 248 in a direction away from first arm 242. FIG. 5B shows a first state, in which button 248 is maintained, by resilient biasing element 250, out of channel 246. In some non-limiting embodiments or aspects, channel 246 is configured such that, even with button 248 in a first (non-depressed) state, fluid conduit 224 may be held within channel, for example by interference fit.

Turning to FIG. 5C, shown is a second state of the non-limiting embodiment or aspect of occlusion device 232 of FIG. 5B in a second state. In such a second state, force exerted by resilient biasing element 250 may be overcome by a user pressing button 248, which can in non-limiting embodiments be a push-button latch, such that button 248 may be reversibly retained against the force of resilient biasing element 250 in a depressed position, in which button 248 causes channel 246 to be narrowed. In some non-limiting embodiments or aspects, in a depressed position (e.g., second state), button 248 may impinge on a wall of fluid conduit 224 and occlude flow therethrough. In some non-limiting embodiments or aspects, occlusion device 232 may be coupled to a dressing, as described herein.

With reference to FIGS. 5D and 5E, shown is a non-limiting embodiment or aspect of occlusion device 232, in which fluid conduit 224 is occluded by virtue of being compressed in a channel 246 formed by a base 240 and a cover 252. In the illustrated non-limiting embodiment or aspect, occlusion device 232 includes base 240, cover 252 which may be connected at an end thereof to base 240, and a channel 246 defined by base 240 and cover 252. In some non-limiting embodiments or aspects, fluid conduit 224 may be held, prior to closing of cover 252, within a portion of channel 246 arranged in base 240 through interference fit. In some non-limiting embodiments or aspects, cover 252 is hingedly connected to base 240, for example with a hinge 254. In some non-limiting embodiments or aspects, channel 246 has, along at least a portion of a length thereof, a diameter that is smaller than a diameter of fluid conduit 224, such that when fluid conduit 224 is received within channel 246, one or more of base 240 and cover 252 impinge on a wall of fluid conduit 224 and occlude flow therethrough. To secure cover 252 to base 240 and occlude flow through fluid conduit 224, one or both of base 240 and cover 252 may include a latching mechanism 256. Those of skill in the art will appreciate that the type of latching mechanism 256 that is used is not limited, so long as the mechanism 256 can secure cover 252 to base 240 in a reversible manner. In some non-limiting embodiments or aspects, occlusion device 232 may be coupled to a dressing, as described herein.

With reference to FIG. 5F, shown is a non-limiting embodiment or aspect of occlusion device 232, in which fluid conduit 224 is occluded by a valve 258. Valve 258 may be formed out of any useful materials, and can include a valve body 260 that can reversibly stop flow through fluid conduit 224. In some non-limiting embodiments or aspects, a connector (for example, connector 30 as shown in FIGS. 1A and 1B) may include, or may be replaced by, valve 258. In some non-limiting embodiments or aspects, valve 258 can divert flow to extension line 234. In some non-limiting embodiments or aspects, valve body 260 can include an actuator 262, to allow manual actuation of valve 258. In some non-limiting embodiments or aspects, valve 258 may be a stopcock, with a rotatable actuator 262 to allow control of fluid flow through valve body 260. In some non-limiting embodiments or aspects, valve 258 is coupled to a dressing as described herein.

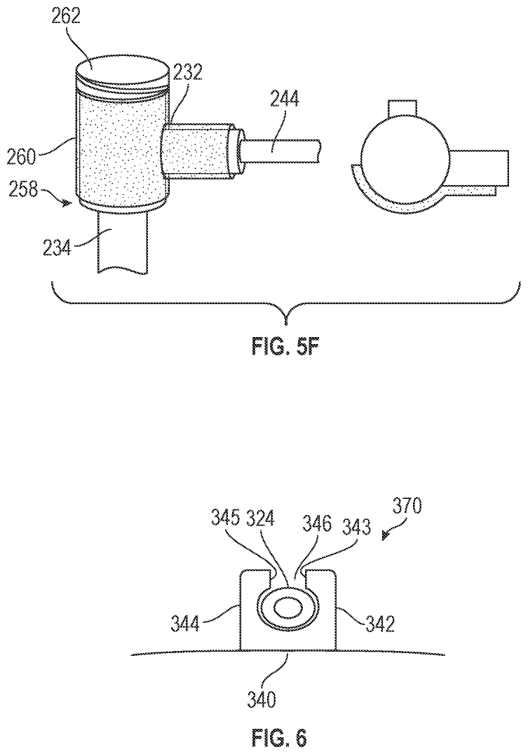

With reference to FIG. 6, shown is a non-limiting embodiment or aspect of a stabilization device 370. Stabilization device may be similar to occlusion devices 232 as described herein, but may be configured such that fluid conduit 324 is held within channel 346, for example by interference fit, but is not occluded thereby. In the illustrated embodiment or aspect, stabilization device 370 includes a base 340 and first and second arms 342, 344 extending from base 340. In some non-limiting embodiments or aspects, first and second arms 342, 344 may extend substantially perpendicularly from base 340, optionally substantially parallel to one another. Base 340 and first and second arms 342, 344 define a channel 346 configured to hold fluid conduit 324, for example by interference fit. Stabilization device 370 as shown in FIG. 6 may be formed of any useful material, and in some non-limiting embodiments or aspects, is formed of a resilient material for example an elastomeric material, to allow for separation of arms 342, 344 (e.g., radially outward away from channel 346) to allow fluid conduit 324 to be introduced into, and removed from channel 346. In some non-limiting embodiments or aspects, first arm 342 and second arm 344 include, at an end separate from base 340, one or more flanges 343, 345. In some non-limiting embodiments or aspects, flanges 343, 345 protrude radially inward, towards one another as shown in FIG. 6, providing a surface that, along with arms 342 and 344, and base 340, holds fluid conduit 324 within channel 346. In some non-limiting embodiments or aspects, stabilization device 370 is coupled to a dressing as described herein.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. An intravascular catheter assembly, comprising:
a catheter adapter comprising:
a distal end;
a proximal end;
a lumen arranged between and in fluid communication with the distal end and the proximal end;
a catheter arranged at the distal end of the catheter adapter and in fluid communication with the lumen; and
a side port in fluid communication with the lumen;
a fluid conduit having a distal end and a proximal end, the distal end of the fluid conduit coupled to the side port;
a connector arranged at the proximal end of the fluid conduit; and
an occlusion device configured to stop flow through the fluid conduit.

2. The intravascular catheter assembly of claim 1, further comprising a dressing having a bottom surface configured to contact a patient's skin and a top surface.

3. The intravascular catheter assembly of claim 2, wherein the occlusion device is coupled to the top surface of the dressing.

4. The intravascular catheter assembly of claim 1, wherein the occlusion device is a clamp comprising a base and first and second arms extending substantially perpendicularly from the base, the base and the first and second arms defining a channel configured to receive the fluid conduit.

5. The intravascular catheter assembly of claim 4, wherein the clamp is formed of an elastomeric material.

6. The intravascular catheter assembly of claim 4, wherein the first arm and the second arm are resiliently biased toward the channel.

7. The intravascular catheter assembly of claim 4, wherein a diameter of the channel is less than a diameter of the fluid conduit, such that when the fluid conduit is received by the channel, fluid flow through the fluid conduit is slowed and/or stopped.

8. The intravascular catheter assembly of claim 1, wherein the occlusion device is a clamp comprising a base and a button defining a channel therebetween, the channel configured to receive the fluid conduit and the button configured to be moveable into the channel, wherein moving the button into the channel causes the clamp to occlude the fluid conduit, thereby slowing and/or stopping fluid flow through the fluid conduit.

9. The intravascular catheter assembly of claim 8, wherein the button is biased away from the channel.

10. The intravascular catheter assembly of claim 9, wherein the button is biased away from the channel by a spring.

11. The intravascular catheter assembly of claim 1, wherein the occlusion device is a clamp comprising:
a base having a first end and a second end;
a cover having a first end and a second end, the first end of the cover hingedly connected to the first end of the base; and
a latching mechanism configured to secure the second end of the base to the second end of the cover,
wherein, when the second end of the base is secured to the second end of the cover, the cover and the base define a channel configured to hold the fluid conduit therein.

12. The intravascular catheter assembly of claim 11, wherein a diameter of the channel is less than a diameter of the fluid conduit, such that when the fluid conduit is held within the channel, fluid flow through the fluid conduit is slowed and/or stopped.

13. The intravascular catheter assembly of claim 1, wherein the occlusion device is a slide clamp comprising a channel having at least one portion with a diameter less than a diameter of the fluid conduit and at least one other portion with a diameter equal to or greater than the diameter of the fluid conduit.

14. The intravascular catheter assembly of claim 1, wherein the occlusion device is arranged between the side port and the connector.

15. The intravascular catheter assembly of claim 1, wherein the occlusion device is a valve.

16. The intravascular catheter assembly of claim 15, wherein the valve is arranged in the connector.

17. The intravascular catheter assembly of claim 15, wherein the valve comprises a manual actuator.

18. The intravascular catheter assembly of claim 1, wherein the connector is a t-connector or a y-connector.

19. The intravascular catheter assembly of claim 1, further comprising a needleless access connector coupled to the connector.

20. The intravascular catheter assembly of claim 19, wherein the needleless access connector is releasably coupled to the connector.
